(19) United States
(12) Patent Application Publication
(10) Pub. No.: US 2017/0370929 A1
(43) Pub. Date: Dec. 28, 2017

(54) POSITIVE ALLOSTERIC MODULATORS OF THE DELTA-OPIOID RECEPTOR

(71) Applicant: BRISTOL-MYERS SQUIBB COMPANY, Princeton, NJ (US)

(72) Inventors: Neil T. Burford, Durham, CT (US); Ying Han, Cheshire, CT (US); Martyn N. Banks, Madison, CT (US); Litao Zhang, Churchville, PA (US); Samuel Gerritz, Guilford, CT (US); Andrew Alt, Durham, CT (US)

(21) Appl. No.: 15/535,652

(22) PCT Filed: Dec. 17, 2015
(86) PCT No.: PCT/US2015/066267

§ 371 Date: Jun. 13, 2017

Related U.S. Application Data
(60) Provisional application No. 62/093,005, filed on Dec. 17, 2014.

(51) Int. Cl. G01N 33/566; A61K 45/06; A61K 31/00

(52) U.S. Cl. CPC G01N 33/566; A61K 45/06; A61K 31/00; G01N 2500/10; G01N 2333/726

(57) ABSTRACT

Described are the discovery, synthesis and pharmacological characterization of δ-opioid receptor-selective positive allosteric modulators (δ PAMs). These δ PAMs may increase the affinity and/or efficacy of the orthosteric agonists leu-enkephalin and SNC80, as measured by β-arrestin recruitment and adenylyl cyclase inhibition. The compounds may be useful pharmacological tools to probe the molecular pharmacology of the δ receptor and to explore the therapeutic potential of δ PAMs in diseases such as chronic pain and depression.

### CROSS REFERENCE TO RELATED APPLICATION

This application claims the priority of U.S. Provisional Application Ser. No. 62/093,005 filed Dec. 17, 2014 which is herein incorporated by reference.

### DESCRIPTION OF THE INVENTION

The δ-opioid receptor is a seven transmembrane domain (7TMD) receptor that belongs to Class A family of G protein coupled receptors (GPCRs). δ Receptor agonists have been shown to be antinociceptive especially in chronic pain models (Gaveriaux-Ruff, C.; Kieffer, B. L., Delta opioid receptor analgesia: recent contributions from pharmacology and molecular approaches. Behavioural pharmacology 2011, 22 (5-6), 405-14) and to have potential as antidepressant agents (Lutz, P. E.; Kieffer, B. L., Opioid receptors: distinct roles in mood disorders. Trends in neurosciences 2013, 36 (3), 195-206). The potential dual effects of δ receptor agonists to alleviate chronic pain and mitigate emotional disorders provide a particularly attractive therapeutic strategy because of the high level of comorbidity between chronic pain and depression. However, directly acting δ receptor agonists suffer from the disadvantage that they can show pro-convulsant effects in animal models, including non-human primates. Indeed, it has been proposed that these seizurogenic properties of δ receptor agonists may be responsible for their antidepressant-like activity analogous to electro-convulsive therapy (Broom, D. C.; Nitsche, J. F.; Pintar, J. E.; Rice, K. C.; Woods, J. H.; Traynor, J. R., Comparison of receptor mechanisms and efficacy requirements for delta-agonist-induced convulsive activity and antinociception in mice. The Journal of pharmacology and experimental therapeutics 2002, 303 (2), 723-9). On the other hand, slowing the rate of administration of the δ receptor agonist SNC80 reduces seizurogenic activity but has no effect on anti-depressant-like effects (Jutkiewicz, E. M.; Rice, K. C.; Traynor, J. R.; Woods, J. H., Separation of the convulsions and antidepressant-like effects produced by the delta-opioid agonist SNC80 in rats. Psychopharmacology 2005, 182 (4), 588-96). Also, some δ receptor agonists (e.g. ADL5859) show no seizures in rat or mouse models (Le Bourdonnec, B.; Windh, R. T.; Ajello, C. W.; Leister, L. K.; Gu, M.; Chu, G. H.; Tuthill, P. A.; Barker, W. M.; Koblish, M.; Wiant, D. D.; Graczyk, T. M.; Belanger, S.; Cassel, J. A.; Feschenko, M. S.; Brogdon, B. L.; Smith, S. A.; Christ, D. D.; Derelanko, M. J.; Kutz, S.; Little, P. J.; DeHaven, R. N.; DeHaven-Hudkins, D. L.; Dolle, R. E., Potent, orally bioavailable delta opioid receptor agonists for the treatment of pain: discovery of N,N-diethyl-4-(5-hydroxyspiro[chromene-2,4′-piperidine]-4-yl)benzamide (ADL5859). Journal of medicinal chemistry 2008, 51 (19), 5893-6). These findings suggest that the convulsive properties of δ receptor agonists can be separated from their anti-depressant effects (Jutkiewicz, E. M.; Baladi, M. G.; Folk, J. E.; Rice, K. C.; Woods, J. H., The convulsive and electroencephalographic changes produced by nonpeptidic delta-opioid agonists in rats: comparison with pentylenetetrazol. The Journal of pharmacology and experimental therapeutics 2006, 317 (3), 1337-48: and Chu Sin Chung, P.; Kieffer, B. L., Delta opioid receptors in brain function and diseases. Pharmacology & therapeutics 2013, 140 (1), 112-20).

Allosteric modulators for GPCRs bind to a site on the receptor that is distinct from the site that binds the orthosteric (or endogenous) agonist. Positive allosteric modulators (PAMs) increase the affinity and/or efficacy of bound orthosteric agonist ligands. One way to measure this allosteric cooperativity from functional assays is to use the operational model (Leach, K.; Sexton, P. M.; Christopoulos, A., Allosteric GPCR modulators: taking advantage of permissive receptor pharmacology. Trends in pharmacological sciences 2007, 28 (8), 382-9, which can quantitate the binding affinity of the allosteric ligand to the free receptor (pK_{B}), the allosteric cooperativity factor (αβ) as well as any intrinsic efficacy “agonism” (τ_{B}) that the allosteric ligand may demonstrate. PAMs have a number of advantages over orthosteric ligands (Christopoulos, A.; Kenakin, T., G protein-coupled receptor allosterism and complexing. Pharmacological reviews 2002, 54 (2), 323-74; May, L. T.; Leach, K.; Sexton, P. M.; Christopoulos, A., Allosteric modulation of G protein-coupled receptors. Annu Rev Pharmacol Toxicol 2007, 47, 1-51; Burford, N. T.; Traynor, J. R.; Alt, A., Positive allosteric modulators of the mu-opioid receptor: a novel approach for future pain medications. British journal of pharmacology 2014). In particular, PAMs can maintain the temporal and spatial fidelity of endogenous receptor activation in vivo. A PAM with little or no intrinsic efficacy (τ_{B}) binds to the target receptor but remains effectively inactive until the endogenous orthosteric agonist is presented to the receptor, upon which the PAM can enhance the cellular response to this native signaling molecule. Therefore, PAMs can amplify the effect of endogenous signaling molecules without disrupting normal physiological regulation of the specific localization and timing of receptor activation, and might therefore be expected to exhibit superior efficacy and side-effect profiles compared to traditional orthosteric agonists. Studies with δ receptor selective ligands, or utilizing a genetic deletion of the δ receptor suggest that native opioid peptide signaling at the δ receptor mediates an increase in pain threshold in models of chronic pain and has antidepressant-like activity in rodent models (Pradhan, A. A.; Befort, K.; Nozaki, C.; Gaveriaux-Ruff, C.; Kieffer, B. L., The delta opioid receptor: an evolving target for the treatment of brain disorders. Trends in pharmacological sciences 2011, 32 (10), 581-90). Therefore, a PAM acting at the δ receptor might be expected to enhance responses to the endogenous agonist peptides and thereby be therapeutically efficacious. In addition, the finite nature of the agonist potency shift (defined by the allosteric cooperativity factor), which saturates when the allosteric site is fully occupied, may increase the safety margin between therapeutic effect and possible side-effects associated with over-activation of the target receptor. Finally, and pertinent to the δ-receptor system which is known to exhibit ligand-biased signaling (Pradhan, A. A.; Smith, M. L.; Kieffer, B. L.; Evans, C. J., Ligand-directed signaling within the opioid receptor family. British journal of pharmacology 2012, 167 (5), 960-9), PAMs can modulate the signaling bias of receptor activation toward desired pathways (Kenakin, T.; Christopoulos, A., Signaling bias in new drug discovery: detection, quantification and therapeutic impact. Nature reviews 2013, 12 (3), 205-16). Thus, δ PAMs may provide a greater therapeutic window between pain relieving and antidepressant-like effects and proconvulsive activity, compared with traditional δ receptor orthosteric agonists.

In this disclosure, the synthesis and structure activity relationship (SAR) of δ PAMs are described. A preferred compound, COMPOUND A was further characterized in a range of cellular functional assays.

### DETAILED DESCRIPTION OF THE INVENTION

The invention is specifically described herein with respect to certain compounds, shown in FIG. 1, which are presented for purposes of exemplification. The application of the invention is not intended to be limited in scope to the exemplified compounds. Instead, the application of the invention is intended to cover any compounds which function to provide the desirable aspects of the invention. In particular, compounds to which the invention may be applicable include any compounds which function to bind to the delta opioid receptors and enhance the binding affinity or efficacy (or both) of an orthosteric agonist.

Discovery and Structure-Activity Relationship (SAR) of δ Receptor PAMs

The δ PAM chemotype was identified from a High Throughput Screen (HTS) using a β-arrestin recruitment assay in a PathHunter U2OS cell line coexpressing μ and δ receptors (U2OS-OPRM1D1) (DiscoveRx, Freemont, Calif.) (Bassoni, D. L.; Raab, W. J.; Achacoso, P. L.; Loh, C. Y.; Wehrman, T. S., Measurements of beta-arrestin recruitment to activated seven transmembrane receptors using enzyme complementation. Methods in molecular biology (Clifton, N.J. 2012, 897, 181-203; Zhao, X.; Jones, A.; Olson, K. R.; Peng, K.; Wehrman, T.; Park, A.; Mallari, R.; Nebalasca, D.; Young, S. W.; Xiao, S. H., A homogeneous enzyme fragment complementation-based beta-arrestin translocation assay for high-throughput screening of G-protein-coupled receptors. Journal of biomolecular screening 2008, 13 (8), 737-47). The screen was executed in PAM mode (in the presence of an EC₁₀ concentration of both endomorphin-I (a μ receptor-selective agonist), and leu-enkephalin which in this assay and cell line was a relatively selective agonist for the δ receptor (Burford, N. T.; Wehrman, T.; Bassoni, D.; O'Connell, J.; Banks, M.; Zhang, L.; Alt, A., Identification of Selective Agonists and Positive Allosteric Modulators for micro- and delta-Opioid Receptors from a Single High-Throughput Screen. Journal of biomolecular screening 2014, 19 (9), 1255-65). Typically, when screening for PAMs, an EC₂₀₋₄₀ concentration of orthosteric agonist is used (Burford, N. T.; Watson, J.; Bertekap, R.; Alt, A., Strategies for the identification of allosteric modulators of G-protein-coupled receptors. Biochem Pharmacol 2011, 81 (6), 691-702). However, in this HTS, the sum of the two EC₁₀ concentrations of agonists offered a compromise between the detection of both μ and δ receptor PAMs and the ability to maintain the overall signal window so that lower efficacy partial agonists could also be detected. Follow-up in vitro testing to determine structural features necessary for PAM activity was performed utilizing CHO-PathHunter cell-lines (CHO-OPRD1 and CHO-OPRM1) obtained from DiscoveRx. Unlike the U2OS cell lines, where forskolin was relatively ineffective at stimulating adenylate cyclase activity, the recombinant CHO cell lines allowed us to investigate both β-arrestin recruitment and inhibition of forskolin-stimulated cAMP accumulation in the same cell line. Concentration response curves (CRCs) for HTS hits were determined both in agonist mode (in the absence of orthosteric agonist) to determine agonist activity of the test compounds, and in PAM mode (in the presence of an EC₂₀ concentration of orthosteric agonist) to determine allosteric modulator activity using the β-arrestin recruitment assays. Compound 7 (Table 1) was identified as a δ PAM.

As shown in Scheme 1, we synthesized a series of close analogs of 7 to optimize δ PAM potency and selectivity. None of the compounds exhibited significant agonist activity, but all of the compounds produced measurable PAM activity at the δ receptor. Analog (1) with an unsubstituted benzyl ring acted as a δ PAM with an EC₅₀ value of 0.2 μM and showed 30-fold selectivity in the β-arrestin recruitment assay compared with PAM activity at the μ receptor. Introduction of a methyl group in various positions around the phenyl ring (2-4) led to the observation that ortho substitution increased δ receptor PAM activity by an order of magnitude, with minimal effect on μ receptor PAM activity, while meta and para substitution did not significantly affect δ or μ receptor PAM activity. The corresponding ortho-F analog was not significantly more active than 1, suggesting that the increased δ receptor activity with the o-methyl was due to a steric rather than an electronic effect. Similarly, the meta- and para-F analogs did not afford an increase in δ receptor activity. Interestingly, even though the ortho-Cl analog (8) was 10-fold less active than the methyl analog (2) at the δ receptor, it showed no PAM activity at the μ receptor up to the highest concentration tested (10 μM). Introduction of a second Cl-group in the meta position (9) provided a modest improvement in δ receptor activity while maintaining selectivity. A more pronounced effect was observed with the ortho-Br analog 10 which produced equipotent PAM activity to 2 at the δ receptor, but no observable PAM activity at the μ receptor. Thus 10 (COMPOUND B) was the most δ receptor-selective analog we have identified to date. The effect of ortho substitution on δ receptor PAM potency and selectivity appears to be restricted to small substituents. As shown with analogs 11-15, larger ortho substituents did not improve δ PAM activity and had no effect on selectivity. Similarly, more drastic changes to the chemotype, such as increasing the chain length between the ether oxygen and the phenyl ring, or replacement of the benzyl ether with a phenyl amide, yielded a significant loss in δ receptor PAM activity. The most potent δ PAM identified was 2 (COMPOUND A), which in the presence of an EC₂₀ of leu-enkephalin, produced a β-arrestin response with an average EC₅₀ of 33 nM in CHO-OPRD1 cells (Table 1). Representative agonist and PAM mode CRCs for COMPOUND A at the μ and δ receptor are shown in FIG. 1. In this example, COMPOUND A produced little or no activity in agonist mode, but in PAM mode (in the presence of an EC₂₀ of leu-enkephelin (in CHO-OPRD1 cells) or endomorphin-1 (in CHO-OPRM1 cells)) produced a δ PAM mode response with an EC₅₀ of 48 nM in CHO-OPRD1 cells and 2 uM in CHO-OPRM1 cells.

### Functional Characterization of COMPOUND A

COMPOUND A was further characterized in several functional assays. In the CHO-OPRD1 PathHunter cells, COMPOUND A effects on leu-enkephalin potency and efficacy were studied in both β-arrestin recruitment assays and inhibition of forskolin-stimulated cAMP accumulation assays. In the β-arrestin recruitment assay, COMPOUND A (up to 10 μM) produced only marginal agonist activity (˜10% of a maximal response to leu-enkephalin) and produced a robust 18-fold leftward shift in the potency of leu-enkephalin (FIG. 2A). A small increase in the efficacy of the response, relative to leu-enkephalin alone, was also observed. This suggests that COMPOUND A is PAM with little or no intrinsic efficacy in this system. In the cAMP assay, COMPOUND A produced robust agonist activity resulting in full agonism at concentrations above 3 μM (FIG. 2B). At lower concentrations, COMPOUND A produced leftward shifts in the CRC for leu-enkephalin. At a 370 nM concentration of COMPOUND A (the highest concentration at which a potency for leu-enkephalin could be determined) the potency of leu-enkephalin was increased by 56-fold. This suggests that COMPOUND A is a PAM-agonist in this system (Christopoulos, A.; Changeux, J. P.; Catterall, W. A.; Fabbro, D.; Burris, T. P.; Cidlowski, J. A.; Olsen, R. W.; Peters, J. A.; Neubig, R. R.; Pin, J. P.; Sexton, P. M.; Kenakin, T. P.; Ehlert, F. J.; Spedding, M.; Langmead, C. J., International union of basic and clinical pharmacology. XC. multisite pharmacology: recommendations for the nomenclature of receptor allosterism and allosteric ligands. Pharmacological reviews 2014, 66 (4), 918-47). The difference in observed agonist activity for COMPOUND A in CHO-OPRD1 cells between the β-arrestin recruitment assay and the cAMP assay may reflect a higher level of signal amplification and thus a higher receptor reserve in the cAMP assay compared to the β-arrestin recruitment assay (Ehlert, F. J., Analysis of allosterism in functional assays. The Journal of pharmacology and experimental therapeutics 2005, 315 (2), 740-54; Kenakin, T.; Watson, C.; Muniz-Medina, V.; Christopoulos, A.; Novick, S., A simple method for quantifying functional selectivity and agonist bias. ACS chemical neuroscience 2012, 3 (3), 193-203). Similar findings were observed using the small molecule orthosteric agonist SNC80 (Table 2). Using an operational model of allosterism (Leach, K.; Sexton, P. M.; Christopoulos, A., Allosteric GPCR modulators: taking advantage of permissive receptor pharmacology. Trends in pharmacological sciences 2007, 28 (8), 382-9) (see Methods and Materials), composite cooperativity (αβ) values and pK_{B} values were determined for COMPOUND A across these assays and different orthosteric agonists (Table 2). In this case, the pK_{B} values denote the equilibrium dissociation binding constant for COMPOUND A at the δ receptor in the absence of orthosteric agonist (i.e. at the free receptor).

An ordinary expert in the art would expect that the pK_{B} values should be the same across all the functional assays, and orthosteric agonists used, since the pK_{B} represents the binding affinity of COMPOUND A to the free receptor. Two way ANOVA with multiple comparison test was calculated from the pK_{B} values in Table 2. No significant difference was observed for pK_{B} values between the different orthosteric agonist ligands used in the same functional assay. For SNC80 there was also no significant difference in pKb values across the different functional pathways tested. However, for leu-enkephalin there was a significant difference in the pK_{B} values between β-arrestin recruitment and cAMP inhibition (p<0.001). This difference in pK_{B} value was surprising and may have been attributed to the increasing level of agonist activity of COMPOUND A observed at higher concentrations of COMPOUND A. This reduces the signal to noise window of the assay and increases the error for determining an accurate EC₅₀ of the orthosteric agonist, particularly at higher concentrations of COMPOUND A. In many instances the pK_{A} values were fixed to the reported equilibrium dissociation constants for the orthosteric agonists to obtain meaningful data and this may impact the overall values for the other parameters (Table 2). In some cases the allosteric effect did not reach a plateau (i.e. the allosteric EC₅₀ shift did not reach a ceiling effect before full agonism was observed with COMPOUND A, or the highest concentration of COMPOUND A used was not a saturating concentration and did not cause the allosteric EC₅₀ shift to reach its ceiling), making accurate assessment of the allosteric parameters more prone to error. However, all available data suggests that COMPOUND A is a δ PAM or a δ PAM-agonist in these functional assays, in this cell line which expresses recombinant δ receptors. COMPOUND A or its analogs may behave as PAMs alone, or have significant agonist activity in cells or tissues expressing endogenous levels of δ receptors.

The observation that COMPOUND A produced PAM activity in the functional assays at concentrations far below the calculated K_{B} would initially suggest that COMPOUND A is only occupying a small fraction of the receptors at these concentrations. However, one must remember the reciprocal nature of affinity modulation which states that the affinity of a PAM in the presence of orthosteric agonist is defined by K_{B}/α. Therefore, a larger fraction of the receptors are occupied by COMPOUND A at these lower concentrations when in the presence of bound orthosteric agonist.

In accordance with the present invention, we have identified and characterized δ receptor-selective PAMs including, for example, COMPOUND A. By virtue of the present invention, this class of compounds may make it possible to treat a variety of diseases such as, for example, chronic pain, depression and other therapeutic indications.

### Methods and Materials

### Chemistry

Commercially available analogs were purchased or synthesized according to Scheme 1 (2, 6, 8-15). All purchased and newly synthesized analogs provided analytical data consistent with their assigned structures.

### Synthesis of Intermediate A (Scheme 1):

To a solution of 4-hydroxybenzaldehyde (1.5 g, 12.28 mmol) in 2-propanol (35 ml) was added 5,5-dimethylcyclohexane-1,3-dione (3.44 g, 24.57 mmol) and H₂SO₄ (98%, 0.098 ml, 1.842 mmol). The reaction mixture was refluxed for 1.5 hours in an oil bath and then cooled to room temperature, forming a white precipitate. After filtration, 3 grams of 9-(4-hydroxyphenyl)-3,3,6,6-tetramethyl-3,4,5,6,7,9-hexahydro-1H-xanthene-1,8(2H)-dione was obtained in 65% yield (98% purity by LCMS analysis). ¹H NMR (400 MHz, CD₃Cl) δ 7.09 (d, J=8.6 Hz, 2H), 6.56 (d, J=8.6 Hz, 2H), 4.67 (s, 1H), 2.46 (s, 4H), 2.23 (s, 2H), 2.21 (s, 2H), 1.10 (s, 6H), 1.00 (s, 6H); ESI-MS m/z=367.08 [M+H]+.

### Synthesis of Analogs 1-15:

General Procedure. To a solution of 9-(4-hydroxyphenyl)-3,3,6,6-tetramethyl-3,4,5,6,7,9-hexahydro-1H-xanthene-1,8(2H)-dione (100 μmol, 36.6 mg) in DMF (1.2 mL) was added ArCH₂Br (200 μmol) and Cs₂CO₃ (65.2 mg, 200 μmol). The reaction mixture was stirred at room temperature overnight. 10 μL of the reaction solution was taken, dissolved in MeOH (0.2 mL) and analyzed by LCMS. The LCMS showed that the reaction was complete and the desired product as a major peak was found. The product was purified via preparative LC/MS with the following conditions: Column: XBridge C18, 19×200 mm, 5-μm particles; Mobile Phase A: 5:95 acetonitrile: water with 10 mM ammonium acetate; Mobile Phase B: 95:5 acetonitrile: water with 10 mM ammonium acetate; Gradient: 70-100% B over 15 minutes, then a 5-minute hold at 100% B; Flow: 20 mL/min. Fractions containing the desired product were combined and dried via centrifugal evaporation.

Two analytical LC/MS injections were used to determine the final purity: Injection 1 conditions: Column: Waters BEH C18, 2.0×50 mm, 1.7-μm particles; Mobile Phase A: 5:95 acetonitrile: water with 10 mM ammonium acetate; Mobile Phase B: 95:5 acetonitrile: water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm.

Injection 2 conditions: Column: Waters BEH C18, 2.0×50 mm, 1.7-μm particles; Mobile Phase A: 5:95 methanol: water with 10 mM ammonium acetate; Mobile Phase B: 95:5 methanol: water with 10 mM ammonium acetate; Temperature: 50° C.; Gradient: 0% B, 0-100% B over 3 minutes, then a 0.5-minute hold at 100% B; Flow: 0.5 mL/min; Detection: UV at 220 nm.

Proton NMR was acquired in deuterated CDCl₃ or DMSO.

### 3,3,6,6-tetramethyl-9-(4-((2-methylbenzyl)oxy)phenyl)-3,4,5,6,7,9-hexahydro-1H-xanthene-1,8(2H)-dione (2, COMPOUND A)

¹H NMR (400 MHz, CHLOROFORM-d) δ 7.51-7.35 (m, 2H), 7.26-7.18 (m, 4H), 6.89 (dd, J=14.2, 8.6 Hz, 2H), 5.05 (s, 2H), 4.72 (s, 1H), 2.49 (d, J=5.9 Hz, 4H), 2.38 (d, J=7.8 Hz, 4H), 2.27-2.21 (m, 3H), 1.16-1.10 (m, 6H), 1.07-1.00 (m, 6H). HRMS: Cal. C31 H35 O4=471.2530, found: 471.2538

### 9-(4-((2-bromobenzyl)oxy)phenyl)-3,3,6,6-tetramethyl-3,4,5,6,7,9-hexahydro-1H-xanthene-1,8(2H)-dione (10, COMPOUND B)

The yield of the product was 20.6 mg, and its estimated purity by LCMS analysis was 100%.

¹H NMR (500 MHz, DMSO-d6) δ 7.67 (d, J=7.7 Hz, 1H), 7.56 (d, J=7.3 Hz, 1H), 7.42 (t, J=7.5 Hz, 1H), 7.31 (t, J=7.3 Hz, 1H), 7.10 (d, J=8.1 Hz, 2H), 6.88 (d, J=8.4 Hz, 2H), 5.04 (s, 2H), 4.48 (s, 1H), 2.54 (d, J=11.4 Hz, 4H), 2.26 (d, J=16.1 Hz, 2H), 2.09 (d, J=16.1 Hz, 2H), 1.04 (s, 6H), 0.91 (s, 6H). HRMS: Cal. C30 H32 O4 Br=535.1478, found: 535.1478

### Cell Lines:

Chinese Hamster Ovary (CHO) PathHunter® cells expressing enzyme acceptor (EA)-tagged β-arrestin 2 and either ProLink (PK)-tagged δ receptor(CHO-OPRD1), or PK-tagged μ receptor (CHO-OPRM1) were from DiscoveRx (Freemont, Calif.). Cells were grown in F-12 media (Invitrogen 11765), containing Hyclone FBS 10%, Hygromycin 300 μg/mL (Invitrogen 10687) G418 800 μg/mL (Invitrogen 10131) and maintained at 37° C. in a humidified incubator containing 5% CO₂. These cells were used for β-arrestin recruitment assays and inhibition of forskolin-stimulated cAMP accumulation assays described below.

### Materials:

PathHunter® detection reagents were from DiscoveRx™ (Freemont, Calif.). Cell culture media and supplements were from Life Technologies™ (Carlsbad, Calif.). Lance-Ultra cAMP detection reagents were from PerkinElmer Life Sciences (Cambridge, Mass.). Endomorphin-I was obtained from Tocris. All other chemicals, unless otherwise specified, were purchased from Sigma (St. Louis, Mo.).

### PathHunter β-Arrestin Assay

Confluent flasks of CHO-OPRM1 and CHO-OPRD1 cells were harvested with TrypLE™ Express, and resuspended in F-12 media supplemented with 10% FBS and 25 mM HEPES, at a density of 6.67e⁵ cells/ml and plated (3 μL/well) into white solid TC-treated 1536-well plates (Corning, N.Y.). Plates were incubated overnight at 37° C. in a 5% CO₂ humidified incubator. The next day, compounds (40 nL of 100×final concentration in 100% DMSO) were added to cell plates by acoustic dispense using an Echo-550 (Labcyte, Sunnyvale, Calif.) from Echo-qualified 1536-well source plates (Labcyte). Next, 1 μL of assay buffer (agonist mode), or assay buffer containing a low concentration (˜4×EC₂₀) of orthosteric agonist (PAM mode), were added to assay plates. The orthosteric agonists used are described in the Results & Discussion. Plates were lidded and incubated at room temperature for 90 min. Incubations were terminated by the addition of 2 μL PathHunter® Reagent (DiscoveRx). One hour later luminescence was detected using a Viewlux® imaging plate reader (PerkinElmer).

Inhibition of Forskolin-Stimulated cAMP Accumulation Assays.

CHO-OPRD1 cells were grown to confluence (as described above). Cells were harvested and resuspended at 1e⁶ cells/mL in assay buffer (HBSS+25 mM HEPES, +0.05% BSA). Compounds (30 nl of 100×final concentration in 100% DMSO) were added to 1536-well white solid NT plates by acoustic dispense using an Echo-550 followed by a 1 μL addition of cells (2000 cells/well) to all wells. Next, 1 μL of either assay buffer (for agonist mode) or assay buffer containing a 3× EC₂₀ concentration of orthosteric agonist (PAM mode) was added. Finally, 1 μL of 3× Forskolin (2 μM final) was added. Plates were lidded and incubated for 45 min at RT. Incubations were terminated by the addition of Lance-Ultra cAMP detection reagent (Perkin Elmer) (1.5 μL of Eu-cryptate-labelled cAMP tracer in lysis buffer, followed by 1.5 μL of U-light conjugated anti-cAMP antibody in lysis buffer). After a 1 hr incubation at room temperature, time-resolved fluorescence (TRF) was detected on a Viewlux® or Envision® plate reader (PerkinElmer) with excitation at 337 nm and emission reads at 615 nm and 665 nm. The ratiometric data (665 nm read/615 nm read)*10,000 was then converted to cAMP (nM) based on a standard curve for cAMP (replacing the cell addition step) run at the same time and under identical conditions to the assay.

Characterization of δ-opioid receptor-selective PAMs in the CHO-OPRD1 cAMP assay, using curve-shift assays, were performed as described above using orthosteric agonists described in the Results & Discussion.

### Data Analysis

For all experiments data were analyzed and EC₅₀ or Ki values determined using nonlinear regression analysis to fit a logistic equation using GraphPad Prism version 6 (GraphPad, San Diego, Calif.). pK_{B} and αβ values were determined using the “Operational Model of Allosterism”⁸ (see (1) below), using Graphpad Prism version 6.

$$E = \frac{{Em ( {τ}_{A} [ A ] ( {K}_{B} + α β [ B ] ) + {τ}_{B} [ B ] {K}_{A} )}^{n}}{\begin{matrix}{( [ A ] {K}_{B} + {K}_{A} {K}_{B} + {K}_{A} [ B ] + α [ A ] [ B ] )}^{n} + \\ {( {τ}_{A} [ A ] ( {K}_{B} + α β [ B ] ) + {τ}_{B} [ B ] {K}_{A} )}^{n}\end{matrix}} \qquad ( 1 )$$

Within this model, E is the pharmacological effect, K_{A} and K_{B} denote the equilibrium binding constants for the orthosteric ligand, A, and the allosteric ligand, B, at the receptor. The binding cooperativity factor, α represents the effect of the allosteric ligand on orthosteric agonist binding affinity, and vice versa. An activation cooperativity factor, β, denotes the effect the allosteric ligand has on orthosteric agonist efficacy. Agonism constants τ_{A} and τ_{B}, represent the intrinsic activity of the orthosteric agonist and any intrinsic activity of the allosteric ligand, respectively, which is dependent on the cell context and receptor expression level of the cell system, and intrinsic efficacy of the ligands used. The remaining parameters, Em and n, denote the maximal response of the system, and the slope, respectively.

In one aspect of the invention, there is provided a method of screening to identify delta-opioid receptor positive allosteric modulators comprising the steps of:
(a) adding a positive allosteric modulator test compound and a low concentration of a delta-selective orthosteric agonist to cells;
(b) measuring the effect of said delta-selective orthosteric agonist and said test compound on said cells; and
(c) identifying said test compound as being a positive allosteric modulator as evidenced by a decrease in the positive allosteric agonist activity of said test compound.

Preferably, the low concentration of a delta-selective orthosteric agonist is selected from the group consisting of:
(a) less than or equal to about the calculated EC80 in said cells;
(b) less than or equal to about the calculated EC70 in said cells;
(c) less than or equal to about the calculated EC60 in said cells;
(d) less than or equal to about the calculated EC50 in said cells;
(e) less than or equal to about the calculated EC40 in said cells;
(f) less than or equal to about the calculated EC30 in said cells;
(g) less than or equal to about the calculated EC20 in said cells;
(h) less than or equal to about the calculated EC10 in said cells.

In another aspect of the invention, there is provided a method of screening to identify delta-opioid receptor negative allosteric modulators comprising the steps of:
(i) adding a negative allosteric modulator test compound and a high concentration of a delta-selective orthosteric agonist to cells;
(ii) measuring the effect of said delta-selective orthosteric agonist and said test compound on said cells; and
(iii) identifying said test compound as being a negative allosteric modulator as evidenced by a decrease in the negative allosteric agonist activity of said test compound.

Preferably, the low concentration of a delta-selective orthosteric agonist is selected from the group consisting of:

(a) greater than or equal to about the calculated EC10 in said cells;

(b) greater than or equal to about the calculated EC20 in said cells;

(c) greater than or equal to about the calculated EC30 in said cells;

(d) greater than or equal to about the calculated EC40 in said cells;

(e) greater than or equal to about the calculated EC50 in said cells;

(f) greater than or equal to about the calculated EC60 in said cells;

(g) greater than or equal to about the calculated EC70 in said cells;

(h) greater than or equal to about the calculated EC80 in said cells;

(i) greater than or equal to about the calculated EC90 in said cells; and

(j) greater than or equal to about the calculated EC100 in said cells.

In another aspect of the invention, there is provided a method of treating pain in a patient in need thereof comprising administering to the patient a compound which is a positive allosteric modulator for the delta-opioid receptor.

In another aspect of the invention, there is provided a method of treating pain in a patient in need thereof comprising administering to the patient a compound which is a positive allosteric modulator for the delta-opioid receptor in combination with another compound which is an orthosteric agonist for the delta-opioid receptor. Preferably, the compound is selective for delta-opioid receptors over mu-opioid receptors. Preferably, the compound which is a positive allosteric modulator for the delta-opioid receptor and is selective for delta-opioid receptors over mu-opioid receptors. Preferably, the compound is effective to provide augmentation of at least one delta-opioid receptor function selected from G protein activation, inhibition of adenylyl cyclase activity, or b-arrestin recruitment.

In another aspect of the invention, there is provided a method of modulating the delta-opioid receptor comprising contacting the receptor with a compound that is effective to provide an increase in the receptor function in the presence of orthosteric exogenous or endogenous agonist. Preferably, the increase in receptor function is observed in maximal effect, potency, or both.

### BRIEF DESCRIPTION OF THE DRAWINGS

FIG. 1 β-arrestin recruitment response to COMPOUND A in agonist mode (in the absence of orthosteric agonist) and in PAM mode (in the presence of an EC₂₀ of orthosteric agonist) in PathHunter cells expressing δ receptors (CHO-OPRD1) and μ receptors (CHO-OPRM1). For CHO-OPRD1 cells the orthosteric agonist was leu-enkephalin and for CHO-OPRM1 cells the orthosteric agonist was endomorphin-I. In PAM mode, The EC₂₀ response of orthosteric agonist was normalized to 0%. 100% represents the response to a maximally effective concentration of orthosteric agonist. Data are the mean±sem, n=4.

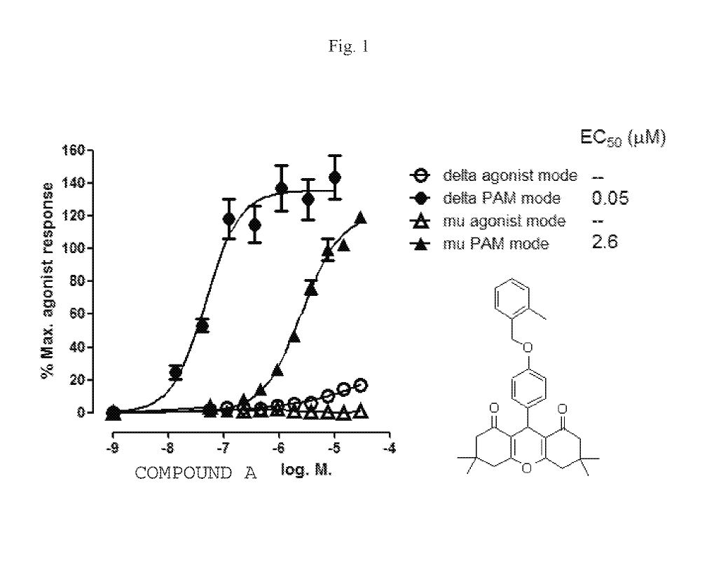

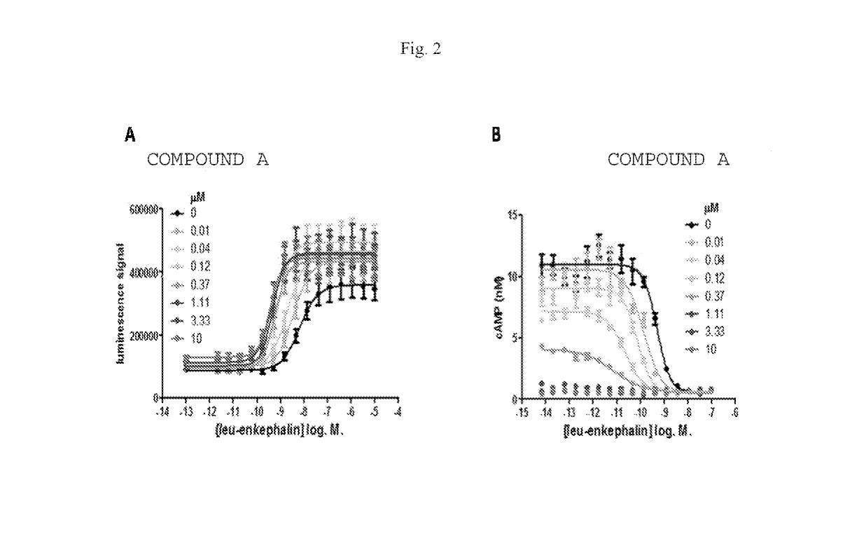

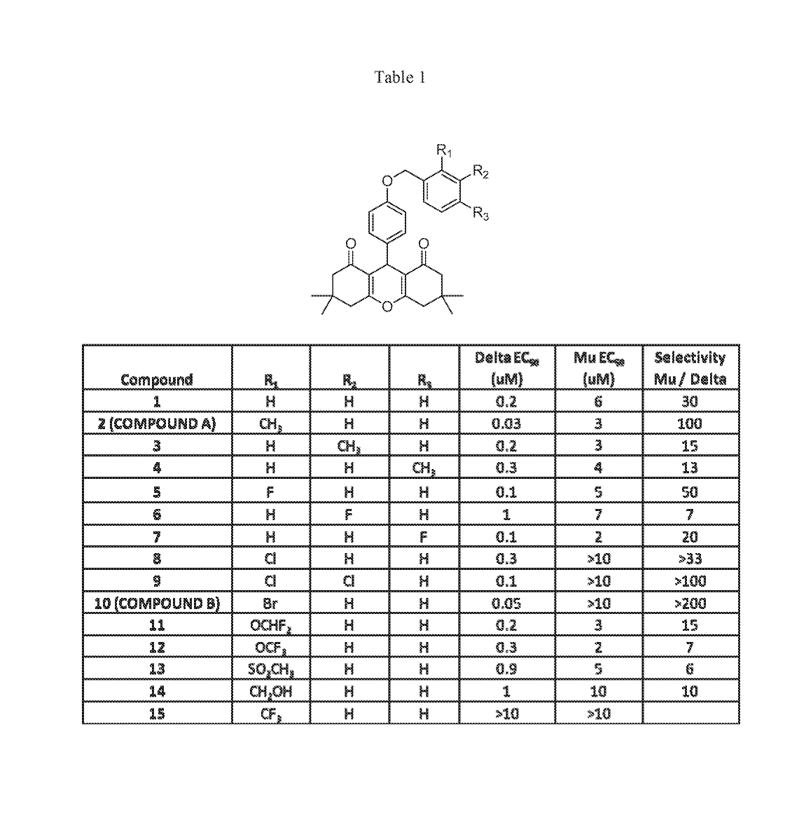

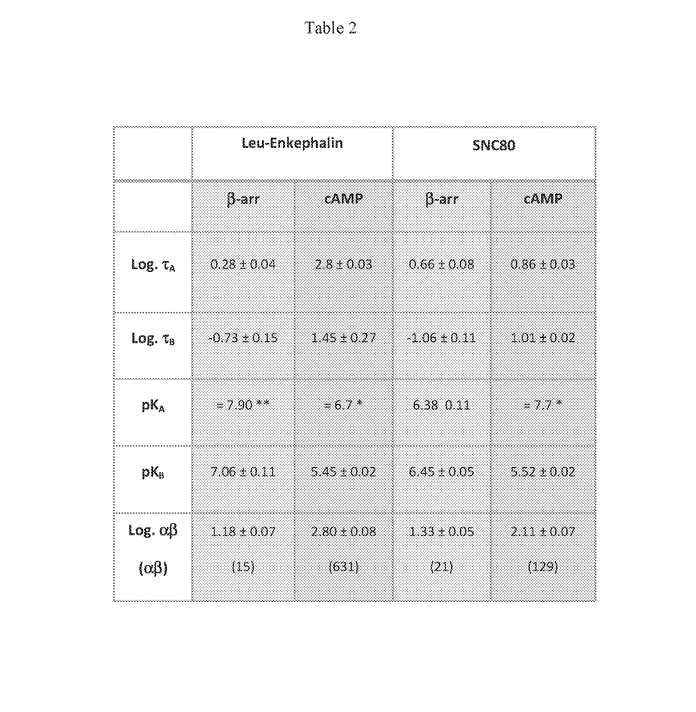

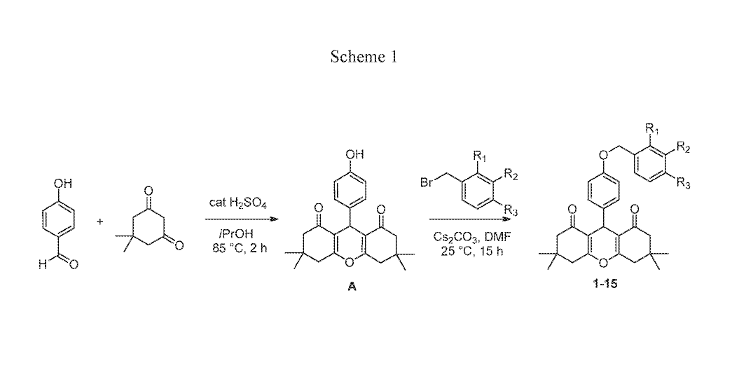

### Table 1

Structure activity relationship of the δ-PAM chemotype in PathHunter CHO-OPRD1 and CHO-OPRM1 cells. No activity was observed in agonist mode (in the absence of orthosteric agonist (data not shown)). In PAM mode (in the presence of an EC₂₀ of leu-enkephalin for OPRD1 cells, or an EC₂₀ of endomorphin-I for OPRM1 cells), robust responses were observed (similar in Eₘₐₓ to the full orthosteric agonist) with the mean EC₅₀ₛ reported in the table (n=3).

FIG. 2 Effect of increasing concentrations of COMPOUND A on leu-enkephalin concentration response curves in □-arrestin recruitment (A), and in inhibition of forskolin-stimulated cAMP accumulation (B), in CHO-OPRD1 cells. Data is the mean±sem, n=4. Data were fitted to the operational model of allosterism (see Table 2).

### Table 2

Allosteric parameters for COMPOUND A at the δ receptor. Values for affinity, efficacy, and allosteric cooperativity for orthosteric ligands and COMPOUND A are derived from the operational model of allosterism. Two different orthosteric agonists were used (Leu-enkephalin and SNC80), in β-arrestin recruitment and cAMP inhibition assays. In the model τ_{A} and τ_{B} represent the efficacy of the orthosteric agonist and allosteric modulator, respectively; pK_{A} and pK_{B} represent the binding affinity of the orthosteric agonist and the allosteric modulator, respectively, to the free receptor; and αβ represents the composite allosteric cooperativity factor. Data represent the mean±sem of 3 to 7 expts.

* pK_{A} is fixed to its equilibrium binding affinity as ligand is a full agonist in all endpoints tested

** the pK_{A} of Leu-enk in endpoints where they are partial agonists was obtained from fitting their CRC to the Operational model of agonism to obtain a functional affinity in each endpoint tested.

1. A method of screening to identify delta-opioid receptor positive allosteric modulators comprising the steps of:
(a) adding a positive allosteric modulator test compound and a low concentration of a delta-selective orthosteric agonist to cells;
(b) measuring the effect of said delta-selective orthosteric agonist and said test compound on said cells; and
(c) identifying said test compound as being a positive allosteric modulator as evidenced by a decrease in the positive allosteric agonist activity of said test compound.

2. The method according to claim 1, wherein the low concentration of a delta-selective orthosteric agonist is selected from the group consisting of:
(a) less than or equal to about the calculated EC80 in said cells;
(b) less than or equal to about the calculated EC70 in said cells;
(c) less than or equal to about the calculated EC60 in said cells;
(d) less than or equal to about the calculated EC50 in said cells;
(e) less than or equal to about the calculated EC40 in said cells;
(f) less than or equal to about the calculated EC30 in said cells;
(g) less than or equal to about the calculated EC20 in said cells;
(h) less than or equal to about the calculated EC10 in said cells.

3. A method of screening to identify delta-opioid receptor negative allosteric modulators comprising the steps of:
(i) adding a negative allosteric modulator test compound and a high concentration of a delta-selective orthosteric agonist to cells;
(ii) measuring the effect of said delta-selective orthosteric agonist and said test compound on said cells; and
(iii) identifying said test compound as being a negative allosteric modulator as evidenced by a decrease in the negative allosteric agonist activity of said test compound.

4. The method according to claim 3, wherein the low concentration of a delta-selective orthosteric agonist is selected from the group consisting of:
(a) greater than or equal to about the calculated EC10 in said cells;
(b) greater than or equal to about the calculated EC20 in said cells;
(c) greater than or equal to about the calculated EC30 in said cells;
(d) greater than or equal to about the calculated EC40 in said cells;
(e) greater than or equal to about the calculated EC50 in said cells;
(f) greater than or equal to about the calculated EC60 in said cells;
(g) greater than or equal to about the calculated EC70 in said cells;
(h) greater than or equal to about the calculated EC80 in said cells;
(i) greater than or equal to about the calculated EC90 in said cells; and
(j) greater than or equal to about the calculated EC100 in said cells.

5. A method of treating pain in a patient in need thereof comprising administering to the patient a compound which is a positive allosteric modulator for the delta-opioid receptor.

6. A method of treating pain in a patient in need thereof comprising administering to the patient a compound which is a positive allosteric modulator for the delta-opioid receptor in combination with another compound which is an orthosteric agonist for the delta-opioid receptor.

7. The method of claim 6 wherein the compound is selective for delta-opioid receptors over mu-opioid receptors

8. The method of claim 7 wherein the compound which is a positive allosteric modulator for the delta-opioid receptor and is selective for delta-opioid receptors over mu-opioid receptors

9. The method of claim 7 wherein the compound is effective to provide augmentation of at least one delta-opioid receptor function selected from G protein activation, inhibition of adenylyl cyclase activity, or b-arrestin recruitment.

10. The method of claim 8 wherein the compound which is a positive allosteric modulator for the mu-opioid receptor and is effective to provide augmentation of at least one delta-opioid receptor function selected from G protein activation, inhibition of adenylyl cyclase activity, or b-arrestin recruitment.

11. A method of modulating the delta-opioid receptor comprising contacting the receptor with a compound that is effective to provide an increase in the receptor function in the presence of orthosteric exogenous or endogenous agonist.

12. The method of claim 11 wherein the increase in receptor function is observed in maximal effect, potency, or both.
